(19) United States
(12) Patent Application Publication
(10) Pub. No.: US 2009/0088855 A1
(43) Pub. Date: Apr. 2, 2009

(54) POSTERIOR ROD CAPTURING SPACER DEVICE AND METHOD

(75) Inventor: Kevin R. Strauss, Columbia, MD (US)

Correspondence Address: CARTER, DELUCA, FARRELL & SCHMIDT, LLP, 445 BROAD HOLLOW ROAD, SUITE 420, MELVILLE, NY, 11747, US

(73) Assignee: K2M, Inc., Leesburg, VA (US)

(21) Appl. No.: 12/240,007

(22) Filed: Sep. 29, 2008

Related U.S. Application Data
(60) Provisional application No. 60/995,534, filed on Sep. 27, 2007.
(60) Provisional application No. 60/999,310, filed on Oct. 17, 2007.

(51) Int. Cl. A61F 2/44; A61B 17/70

(52) U.S. Cl. 623/17.16; 623/17.11; 606/250; 128/898

(57) ABSTRACT

An implantable device includes a spacing member and an elongate member. The elongate member includes a body portion having a first end coupled to the spacing member and a second end having a retention member. In a system for maintaining disc space between adjacent vertebrae, the implantable device is used in conjunction with a spine fixation member. The spine fixation member includes bone anchors and a spinal rod for releasably securing the retention member of the elongate member thereto in order to prevent expulsion of the spacing member from the disc space. Methods of using the system include unilaterally and bilaterally replacing diseased or damaged intervertebral discs from a posterior or transformational approach.

### CROSS-REFERENCE TO RELATED APPLICATIONS

This application claims the benefit of U.S. Provisional Application No. 60/995,534, filed Sep. 27, 2007, and U.S. Provisional Application No. 60/999,310, filed Oct. 17, 2007, the entire contents of which are incorporated herein by reference.

### TECHNICAL FIELD

The present disclosure relates generally to orthopedic spine surgery and in particular to devices, systems, and methods for vertebral body spacing using a posterior or transformational surgical approach.

### DESCRIPTION OF THE RELATED ART

The human spine is comprised of thirty-three vertebrae at birth and twenty-four as a mature adult. The vertebra is made up of the vertebral body and posterior elements, including the spinous process, transverse processes, facet joints, laminae, and pedicles. The vertebral body consists of a cortical shell which surrounds the cancellous center. Between each pair of vertebrae is an intervertebral disc, which maintains the space between adjacent vertebrae and acts as a cushion under compressive, bending and rotational loads and motions. A healthy intervertebral disc has a great deal of water in the nucleus pulposus; the center portion of the disc. The water content gives the nucleus a spongy quality and allows it to absorb spinal stress. Excessive pressure or injuries to the disc can cause injury to the annulus; the outer ring that holds the disc together.

Generally, the annulus is the first portion of the disc that seems to be injured. These injuries are typically in the form of small tears. These tears heal by scar tissue. The scar tissue is not as strong as normal annulus tissue. Over time, as more scar tissue forms, the annulus becomes weaker. Eventually this can lead to damage of the nucleus pulposus. The nucleus begins to lose its water content due to the damage; it begins to dry up. Because of water loss, the discs lose some of their ability to act as a cushion. This can lead to even more stress on the annulus and still more tears as the cycle repeats. As the nucleus loses its water content it collapses, allowing the vertebrae above and below the disc space to move closer to one another. This results in a narrowing of the disc space between the two vertebrae and often times impingement of the nerves branching off the spinal cord. As this shift occurs, the facet joints located at the back of the spine are forced to shift. This shift changes the load distribution and balance at the facet joints affecting the way the facet joints work together and can lead to problems in the facet joints or to a premature breakdown of the joint.

When a disc or vertebrae is damaged due to disease or injury standard practice is to remove part or all of the intervertebral disc, insert a natural or artificial disc spacer and construct an artificial structure to hold the affected vertebrae in place to achieve a spinal fusion. In doing so, while the diseased or injured anatomy is addressed and the accompanying pain is significantly reduced, the natural biomechanics of the spine are affected in a unique and unpredictable way and, more often than not, the patient will develop complicating spinal issues in the future.

To that end, it would be advantageous to treat the disease or injury while maintaining or preserving the natural spine biomechanics. Normal spine anatomy, specifically intervertebral disc anatomy, allows one vertebrae to rotate, with respect to its adjacent vertebrae, about all three axes of the spine. Similarly, the intervertebral disc also allows adjacent vertebrae to translate along all three axes, with respect to one another.

For the above stated reasons, an implantable device which may be used to maintain the disc space between adjacent vertebrae, allow rotation about at least one axis and allow translation about at least one axis, and have a means to prevent expulsion while complementing a posterior stabilizing spinal construct would be helpful. The implantable device would be capable of being introduced into the body using a posterior approach, similar to a PLIF, T-PLIF, or X-PLIF spinal fusion device and may provide a prolonged life span in the body that can withstand early implantation, as is often indicated for younger patients, and will have a limited amount of particulate debris so as to reduce complications over the useful life of the device.

### SUMMARY

An implantable device includes a spacing member and an elongate member. The elongate member includes a body portion having a first end coupled to the spacing member and a second end having a retention member. The spacing member is compressible and may be configured to include dual directional radii along a longitudinal axis and a lateral axis. In embodiments, the spacing member is oblong. The spacing member is configured to translate along at least one axis and rotate about at least one axis.

In a system for maintaining disc space between adjacent vertebrae, the implantable device is used in conjunction with a spine fixation member. The spacing member of the implantable device is adapted for insertion into a disc space between a pair of vertebrae and a spine fixation member is disposed between the pair of vertebrae. In embodiments, the spine fixation member includes bone anchors adapted to be affixed to the vertebrae and a spinal rod which is placed across and secured to the bone anchors. The elongate member of the implantable device is affixed to the spacing member on a first end which is configured to extend out of the disc space. A second end of the elongate member is adapted to be releasably connectable with the spine fixation member in order to prevent expulsion of the spacing member from the disc space.

Methods of using the system, including unilaterally and bilaterally replacing diseased or damaged intervertebral discs from a posterior or transformational approach, are also disclosed. In accordance with the present disclosure, bone anchors are placed in adjacent vertebrae of a patient. A spacing member operably connected to an elongate member is introduced into a disc space opening formed between the adjacent vertebrae. A spinal rod is placed across and secured to the bone anchors and the elongate member is secured thereto. In embodiments utilizing a unilateral vertebral body spacing device, a single spacing member is placed centrally between the adjacent vertebrae. In embodiments utilizing a bilateral vertebral body spacing device, two spacing members are bilaterally placed between the adjacent vertebrae.

### BRIEF DESCRIPTION OF THE DRAWINGS

The foregoing and other features of the present disclosure will become apparent to one skilled in the art to which the present disclosure relates upon consideration of the following description with reference to the accompanying drawings, wherein:

FIG. 1A is a top view of a single level construct with the vertebral body spacing device, placed bilaterally, in accordance with the present disclosure;

FIG. 1B is an end view of the single level construct with the vertebral body spacing device of FIG. 1A;

FIG. 1C is a side view of the single level construct with the vertebral body spacing device of FIG. 1A;

FIG. 2A is a top view of the vertebral body spacing device of FIGS. 1A-1B;

FIG. 2B is an isometric view of the vertebral body spacing device of FIGS. 1A-1B;

FIG. 2C is a cross-sectional view of the vertebral body spacing device of FIGS. 1A-1B;

FIG. 2D is a side view of the vertebral body spacing device of FIGS. 1A-1B;

FIG. 3A is a top view of a single level construct with a vertebral body spacing device, placed centrally, in accordance with the present disclosure;

FIG. 3B is an end view of the single level construct with the vertebral body spacing device of FIG. 3A;

FIG. 3C is a side view of the single level construct with the vertebral body spacing device of FIG. 3A;

FIG. 4A is a top view of the vertebral body spacing device of FIGS. 3A-3B;

FIG. 4B is an isometric view of the vertebral body spacing device of FIGS. 3A-3B;

FIG. 4C is a side view of the vertebral body spacing device of FIGS. 3A-3B;

FIG. 4D is a cross-sectional side view of the vertebral body spacing device of FIGS. 3A-3B;

FIG. 5A is a top view of an alternate embodiment of the spacing member of the vertebral body spacing device in accordance with the present disclosure;

FIG. 5B is a front view of the spacing member of FIG. 5A;

FIG. 5C is a cross-sectional view of the spacing member of FIG. 5B taken along line C-C;

FIG. 6A is a front view of another embodiment of the spacing member of the vertebral body spacing device in accordance with the present disclosure;

FIG. 6B is a front view of the spacing member of FIG. 6A; and

FIG. 6C is a cross-sectional view of the spacing member of FIG. 6B taken along line B-B.

### DETAILED DESCRIPTION OF THE EMBODIMENTS

The vertebral body spacing device of the present disclosure is used in orthopedic spine surgery. A spacing member for maintaining disc space between adjacent vertebrae is coupled with an elongated member which is used to prevent the device from expelling out of the disc space. The device is contemplated as a single, central vertebral body spacing device attached to one spinal rod or as a bilateral device attached to bilaterally placed spinal rods. Either orientation will facilitate a fusion or dynamic stabilization procedure. The former takes advantage of Wolff's Law when a bone graft is introduced. The latter may be used with a posterior dynamic rod system providing anterior column support thereby helping to unload the facet joints and restore balance to the spine.

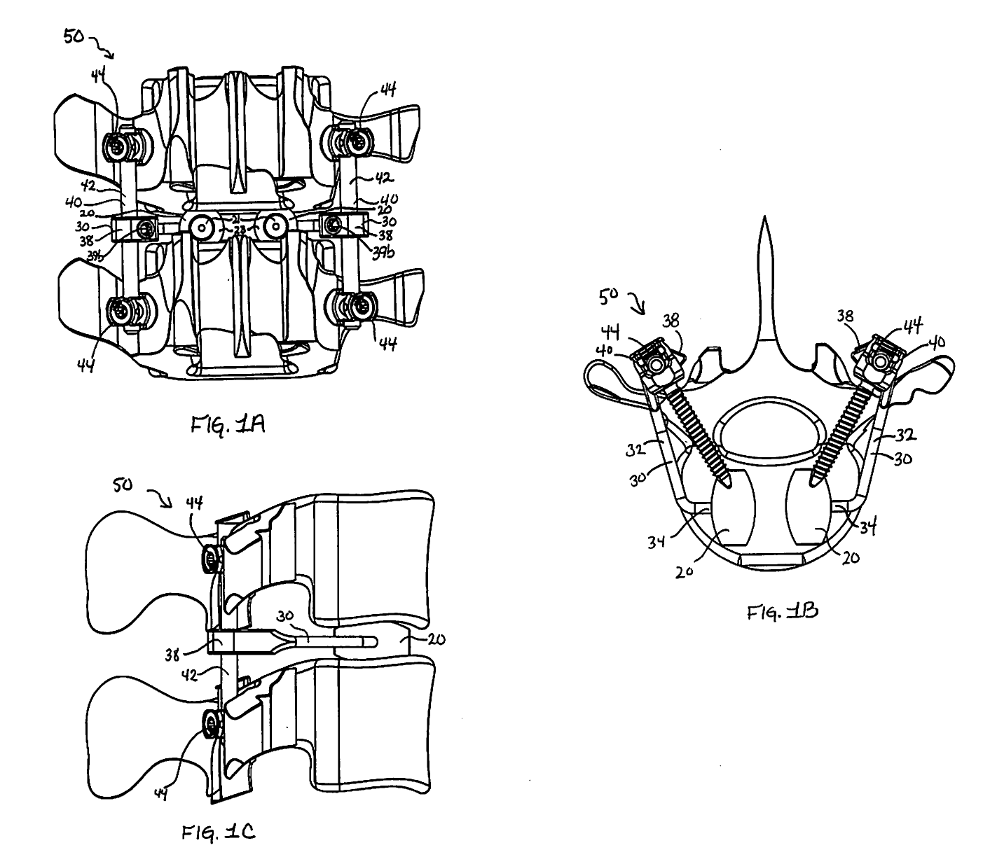

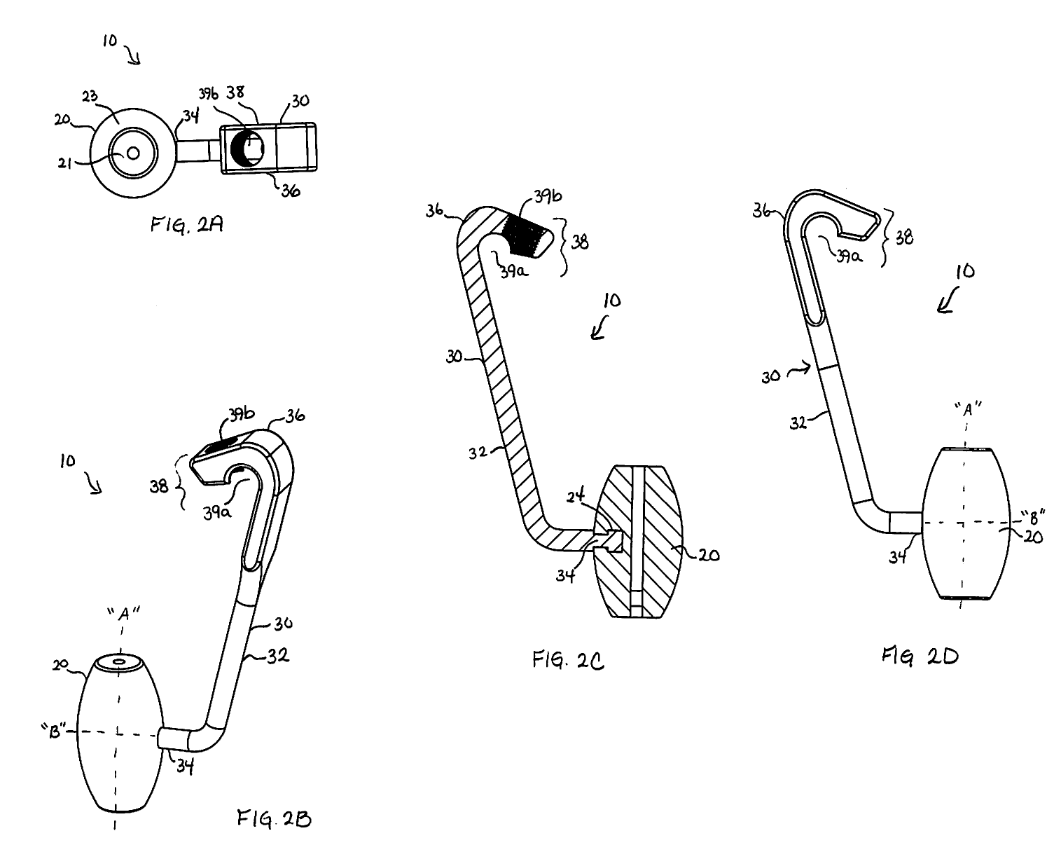

Referring now to the drawings, in which like reference numerals identify identical or substantially similar parts throughout the several views, FIGS. 1A-2D illustrate views of an embodiment of the vertebral body spacing device, placed laterally, in accordance with the principles of the present disclosure. Vertebral body spacing device 10 includes spacing member 20 and elongate member 30.

Spacing member 20 is dimensioned to be positioned between two adjacent vertebrae. Spacing member 20 has sufficient contact surface area with a vertebral body endplate such that minimal subsidence into the endplate occurs and rotation about the longitudinal axis of the spine is possible. As illustrated in the current embodiment, spacing member 20 defines a longitudinal axis “A” and a lateral axis “B” (FIGS. 2B and 2D). Spacing member 20 is elongated along longitudinal axis “A” in the shape of an oblong. The oblong shape provides dual directional radii, one in the longitudinal axis “A” and the other in the lateral axis “B”, along the outer surface of spacing member 20. The dual directional radii of the oblong outer surface of spacing member 20 provides an articulating surface whereby rotation of the vertebrae about the other two axes is achieved. Other shapes are within the purview of those skilled in the art for providing rotational movement to the vertebrae, such as other spherical shapes having two radii of curvature.

Spacing member 20 is also compressible. Material selection may also affect compressibility of spacing member 20. Spacing member 20 may be made of biocompatible materials, including polymeric, metallic, and composites thereof. Polymeric materials include, for example, polyethylene, polypropylene, polyurethane, and polyetheretherketone. Metallic materials may include metals such as surgical grade stainless steel and titanium alloys. In the current embodiment, spacing member 20 may be a composite of a first material 21 within a second material 23. In some embodiments, the first material 21 may provide a soft segment and the second material 23 may provide a hard segment.

The degree of translation, therefore, is affected by the material properties and the geometry of the spacing member 20 as well as by other means within the purview of those skilled in the art. For example, translation along the other two axes may be achieved by sizing spacing member 20 appropriately for the disc space such that the working area of spacing member 20 is within the softer, interior area of the endplate, as it is the cortical rim of the endplate which prevents further translation of one vertebral body to the other.

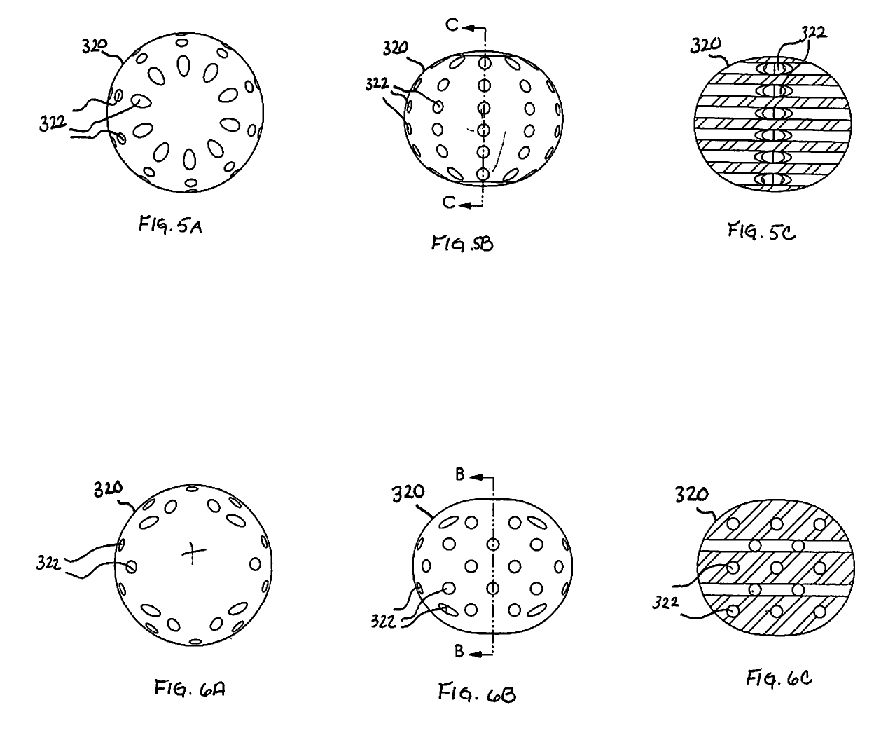

As illustrated in FIGS. 5A-6C spacing member 320 may include voids 322 to allow flexure of the implant in predefined directions. Voids 322 may be generally cylindrical shaped and extend at least partially through spacing member 320. Voids 322 may be radially spaced about spacing member 320 and may be equally or unevenly spaced thereabout. Further, the voids may be any shape within the purview of those skilled in the art. The placement, number, and size of voids 322 within spacing member 320 may be controlled as needed to provide flexibility along at least one predefined axis.

Referring again to FIGS. 1A-2D, spacing member 20 includes opening 24 for coupling with elongate member 30. Opening 24 extends at least partially into spacing member 20 and is configured and adapted to securely receive elongate member 30.

Elongate member 30 is connected to spacing member 20 in order to prevent expulsion of spacing member 20 when positioned in a disc space. Elongate member 30 includes a body portion 32 having a first end 34 and a second end 36. First end 34 of elongate member 30 is coupled with opening 24 of spacing member 20. First end 34 is configured and adapted to fit within opening 24 of spacing member 20. As illustrated in the current embodiment, first end 34 and opening 24 are press fit to couple elongate member 30 and spacing member 20. First end 34 of elongate member 30 may be connected with opening 24 of spacing member 20 through any mechanical and chemical means within the purview of those skilled in the art, such as, for example, interference fitting, press fitting, friction fitting, welding, and adhesive binding.

Second end 36 of elongate member 30 includes retention member 38 for releasably coupling elongate member 30, and thus spacing member 20, to spine fixation member 40. Spine fixation member 40 may be any posterior stabilizing longitudinal member within the purview of those skilled in the art, such a solid spinal rod 42 spanning at least two bone anchors 44. Retention member 38 may be any member capable of securing elongate member 30 to spine fixation member 40. As illustrated in the current embodiment, retention member 38 is a hook 39a and screw 39b set. Other comparable mechanical means for attached the elongate member 30 to spine fixation member 40 are envisioned and within the purview of those skilled in the art.

Elongate member 30 is a semi-rigid device capable of being bent in such a way as to allow implantation and alignment of the device using standard posterior or transforminal approaches and preventing interference with anatomy, such as the exiting nerve root, once implanted, while maintaining the formed shape.

A system 50 for maintaining disc space between adjacent vertebrae utilizes the vertebral body spacing device 10 of the present disclosure in conjunction with a spine fixation device 40 including pedicle screws 44 and rod 42. Any spine fixation member 60, however, adapted to span across adjacent vertebrae and capable of being coupled to vertebral body spacing device 10 may be utilized.

Pedicle screws 44 are bilaterally placed at adjacent vertebrae such that each set of pedicle screws 44 are aligned on one side of the vertebrae. The surgeon modifies the appropriate anatomy in order to access the disc space between the vertebrae for accepting spacing members 20. Spacing members 20 are fitted to the disc space. Elongate members 30, which may be fastened with its respective spacing member 20 via first end 34 prior to implantation or subsequently coupled to spacing member 20 after placement in the disc space, extends out of the disc space and is manipulated for optimal placement of the device. Elongate member 30 may be bent, twisted, curved, straightened, or otherwise shaped to ensure proper securement of spacing member 20 within the disc space, to prevent interference with the existing anatomy of the patient, and to properly align the retention member 38 of the elongate member 30 with spine fixation member 40. Rods 42 are placed across and secured to its respective set of pedicle screws 44 and retention member 38 of the elongate member is secured thereto. In embodiments in which retention member 38 is a hook 39a and screw 39b set, the hook 39a is positioned around rod 42 and screw 39b is placed therethrough to fasten the hook 39a to rod 42. As illustrated the vertebral body spacing devices 10 are placed bilaterally within the vertebrae such that each spacing device 10 is on the same side as the spine fixation member 40 to which it will be fastened.

In some embodiments, before implantation of spacing member 20, a trial device may be attached to a spinal rod and introduced into the disc space for appropriate sizing of the spacing member 20 and to determine if there will be any soft tissue interference by the introduction of the spacing member 20 into the disc space. The trial device may be used to ascertain the amount of contacting surface area of spacing member 20 with the vertebral body endplate to ensure rotational movement of spacing member 20.

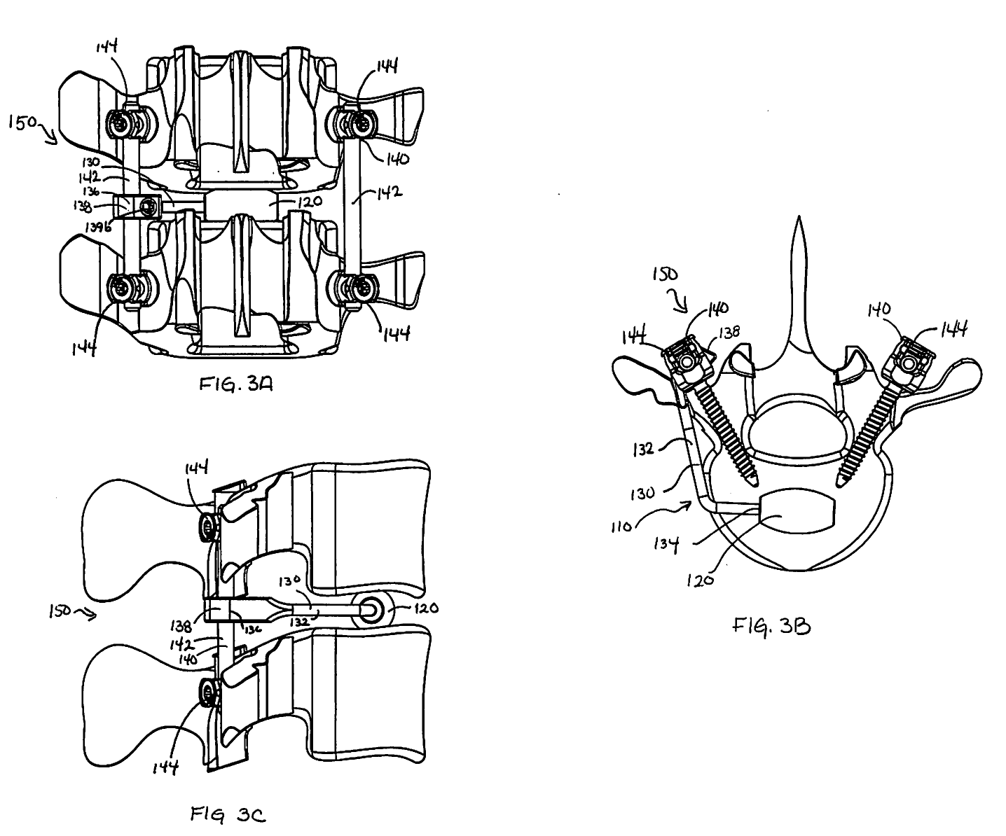

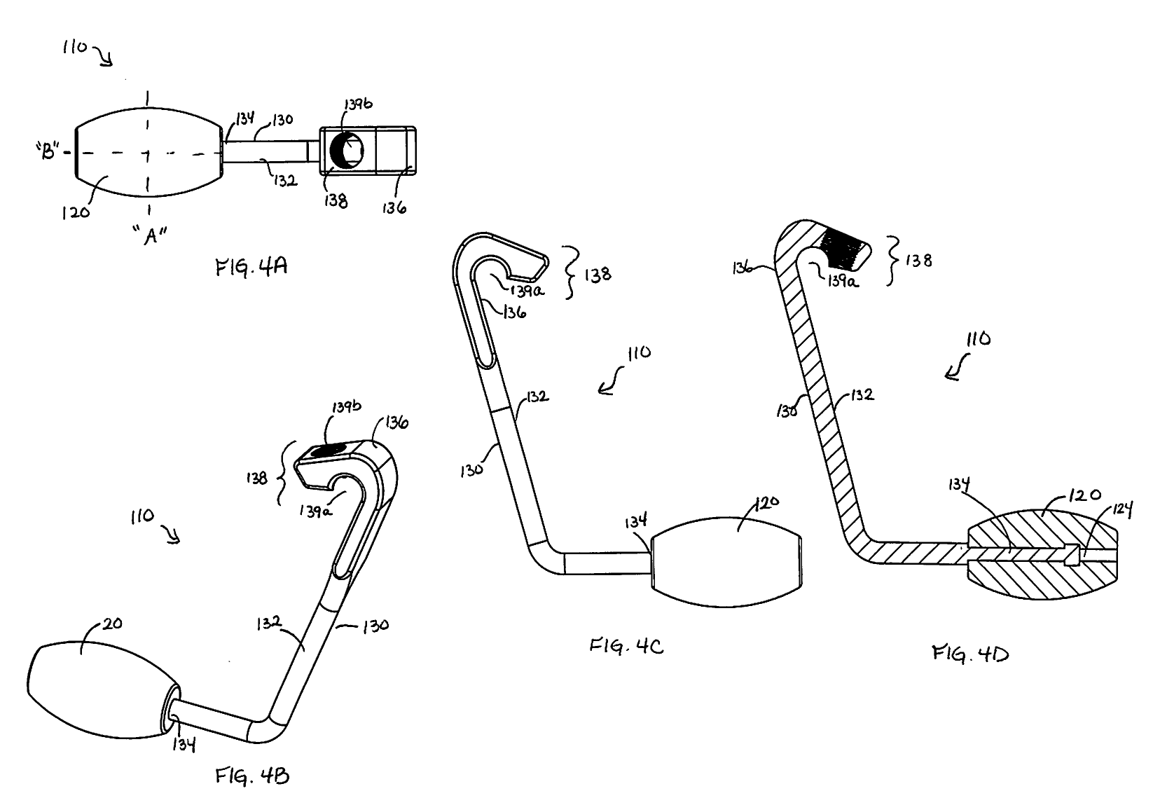

The vertebral body spacing device of the present disclosure is also contemplated to be used as a single, central vertebral body spacing device as illustrated in an alternate embodiment shown in FIGS. 3A-4D. Vertebral body spacing device, shown generally as 110, includes spacing member 120 having opening 124 and elongate member 130 including body portion 132 having first end 134 and second end 136. First end 134 is configured for coupling with opening 124 of spacing member 120 and the second end 136 includes retention member 138 which is adapted to be mechanically fastened to a spine fixation member 140.

In this embodiment, spacing body 120 is elongated along lateral axis “B” to ensure a central fitting and sufficient contact of the working area of the spacing member 120 within the softer, interior area of the endplate between adjacent vertebrae. By providing more contacting surface area of spacing member 120 with the vertebral body endplate, a system 150 for maintaining disc space between adjacent vertebrae utilizing a unilateral vertebral body spacing device 110 may be achieved.

System 150 includes spine fixation device 140 including pedicle screws 144 and rod 142 similar to that described above in FIGS. 1A-2D.

Pedicle screws 144 may be bilaterally placed at adjacent vertebrae in order to provide proper bone fixation between the adjacent vertebrae. Alternatively, a single set of pedicle screws 144 may be placed at adjacent vertebrae. The surgeon modifies the appropriate anatomy in order to access the disc space between the vertebrae for accepting a single spacing member 120. A trial device may be introduced into the disc space for appropriate sizing of spacing member 120 or the spacing member 120 may be directly fitted to the disc space. Elongate member 130, which may be fastened with spacing member 120 via first end 134 prior to implantation or subsequently coupled to spacing member 120 after placement in the disc space, is manipulated for optimal placement of the device. Rods 142 are placed across and secured to its respective set of pedicle screws 44. Retention member 138 of elongate member 130 may be affixed and secured to either rod 142.

Vertebral body spacing device 10 may be fabricated as a single unit or multiple pieces designed for assembly by the surgeon at the time of use. Further, individual components of the vertebral body spacing device 10, i.e. the spacing member 20 and elongate member 30, may likewise be built as a single unit or include more than one piece for assembly. As a single unit, the device or component may be monolithically formed or pre-formed as a composite of multiple pieces. The vertebral body spacing device 10 and unassembled components thereof may be available in a range of sizes to better fit the patient's anatomy and offer greater surgical flexibility.

It will be understood that various modifications may be made to the embodiments disclosed herein. Therefore, the above description should not be construed as limiting, but merely as an exemplification of the embodiments. Those skilled in the art will envision other modifications within the scope and spirit of the present disclosure. Such modifications and variations are intended to come within the scope of the following claims.

What is claimed is:

1. An implantable device comprising:
a spacing member; and
an elongate member including a body portion having a first end coupled to the spacing member and a second end having a retention member.

2. The implantable device according to claim 1, wherein the spacing member is compressible.

3. The implantable device according to claim 1, wherein the spacing member is configured to include dual directional radii along a longitudinal axis and a lateral axis.

4. The implantable device according to claim 3, wherein the spacing member is oblong.

5. The implantable device according to claim 1, wherein the spacing member is configured to translate along at least one axis.

6. The implantable device according to claim 5, wherein the spacing member is configured to translate along three axes.

7. The implantable device according to claim 1, wherein the spacing member is configured to rotate about at least one axis.

8. The implantable device according to claim 7, wherein the spacing member is configured to rotate about three axes.

9. The implantable device according to claim 1, wherein the retention member is a hook.

10. A system for maintaining disc space between adjacent vertebrae comprising:
a spacing member adapted for insertion into a disc space between a pair of vertebrae;
a spine fixation member disposed between a pair of vertebrae; and
an elongate member having a first end and a second end, the first end affixed to the spacing member and configured to extend out of the disc space, the second end adapted to be releasably connectable with the spine fixation member.

11. The system of claim 10, wherein the spine fixation member further comprises bone anchors adapted to be affixed to the vertebrae and a spinal rod configured to be placed across and secured to the bone anchors.

12. The system of claim 10, wherein the spacing member defines a longitudinal axis and is elongated along the longitudinal axis.

13. The system of claim 10, wherein the spacing member defines a lateral axis and is elongated along the lateral axis.

14. A method of maintaining spinal disc space comprising:
placing bone anchors in adjacent vertebrae of a patient;
introducing a spacing member operably connected to an elongate member into a disc space opening formed between the adjacent vertebrae;
extending a spinal rod across the bone anchors; and
securing the elongate member to the spinal rod.

15. The method of claim 14, further comprising the step of:
modifying the anatomy of the patient in order to access the disc space.

16. The method of claim 14, further comprising the step of:
manipulating the elongate member for optimal placement within the patient.

17. The method of claim 14, further comprising the step of:
introducing a trial device into the disc space to determine the optimal sizing of the spacing member.

18. The method of claim 14, wherein the step of introducing the spacing member into a disc space further comprises the step of:
placing the spacing member centrally between the adjacent vertebrae.

19. The method of claim 14, wherein the step of introducing the spacing member into a disc space further comprises the step of:
placing the spacing member within the disc space on the side containing the bone anchors.

20. The method of claim 19, further comprising the steps of:
placing a second set of bone anchors into the adjacent vertebrae such that the first and second sets of bone anchors are bilaterally positioned;
introducing a second spacing member operably connected to an elongate member into the disc space on the side containing the second set of bone anchors;
extending a second spinal rod across the second set of bone anchors; and
securing the second elongate member to the second spinal rod.

21. The method of claim 14, wherein the step of securing the elongate member to the spinal rod further comprises the step of:
fastening a screw within a hook of the elongate member.
